(19) United States
(12) Patent Application Publication
(10) Pub. No.: US 2007/0027189 A1
(43) Pub. Date: Feb. 1, 2007

(54) Use of known active ingredients as radical scavengers

(75) Inventors: Wolfgang-Alexander Simon, Konstanz (DE); Ernst Strum, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/571,570

(22) PCT Filed: Sep. 17, 2004
(86) PCT No.: PCT/EP04/52233

§ 371 Date: Mar. 10, 2006

(30) Foreign Application Priority Data
Sep. 18, 2003 (EP) 0021094.2

(51) Int. Cl. A61K 31/4439

(52) U.S. Cl. 514/338

(57) ABSTRACT

The invention relates to the use of certain proton pump inhibitors in the treatment of pathological manifestations induced or influenced by free radicals.

### TECHNICAL FIELD

The invention relates to the use of compounds from the class of acid secretion inhibitors as radical scavengers.

### PRIOR ART

A whole series of compounds which inhibit gastric acid secretion through blockade of the proton pump and which are therefore referred to as proton pump inhibitors (PPI) are known in the art. These compounds are suitable for the treatment of gastric and intestinal disorders, and some of them are accordingly approved by the health authorities.

### DESCRIPTION OF THE INVENTION

It has now been found that specific proton pump inhibitors whose original area of use is inhibition of acid secretion in the stomach are particularly suitable as radical scavengers. This fact is particularly surprising in as much as the possibility of use as radical scavenger is based on a completely different mechanism than in the case of inhibition of acid secretion. In order to act as inhibitors of acid secretion, the proton pump inhibitors must initially be converted by acid-catalysed rearrangement into a reactive intermediate which then in turn inhibits the proton pump by covalent bonding to the enzyme. The proton pump inhibitors thus do not act per se and are therefore also referred to as prodrugs. This differs for the novel use of the proton pump inhibitors as radical scavengers. In this case, the proton pump inhibitors display their effect without acid activation, that is to say per se, under neutral conditions via the bloodstream, scavenging the radicals which are unwanted for the body by an extracellular route. Because the effect is displayed under neutral conditions, the proton pump inhibitors which are particularly suitable for use as radical scavengers are those having a high stability and thus long half-life under neutral conditions.

The invention thus relates in a first aspect to the use of certain proton pump inhibitors as radical scavengers.

Proton pump inhibitors are designated as those substances which inhibit gastric acid secretion by blocking the proton pump, i.e. which bind covalently to H+/K+-ATPase, the enzyme responsible for gastric acid secretion. This includes in particular active compounds having a 2-[(2-pyridinyl)methyl-sulphinyl]-1H-benzimidazole skeleton or a related skeleton, where these skeletons may be substituted in various forms. According to the invention, the term “proton pump inhibitors” includes not only the active compounds as such, but also their pharmacologically acceptable salts, solvates (in particular hydrates), etc.

Exemplary proton pump inhibitors which may be mentioned are those described and claimed in the following patent applications and patents: DE-A-3531487, EP-A-0 005 129, EP-A-0 124 495, EP-A-0 166 287, EP-A 0 174 726, EP-A-0 184 322, EP-A-0 254 588, EP-A-0 261 478, EP-A-0 268 956, EP-A-0 434 999 and WO-A-9523149. The compounds 2-[2-(N-isobutyl-N-methylamino)benzyl-sulphinyl]benzimidazole (INN: leminoprazole), 2-(4-methoxy-6,7,8,9-tetrahydro-5H-cyclohepta[b]pyridin-9-ylsulphinyl)-1H-benzimidazole (INN: nepaprazole), 2-(4-methoxy-3-methyl-pyridin-2-ylmethyl-sulphinyl)5-pyrrol-1-y-1H-benzimidazole (IY-81149), 5-methoxy-2-[(4-methoxy-3,5-dimethyl-2-pyridinyl)methylsulphinyl]-1H-imidazo[4,5-b]pyridine(tenatoprazole), especially 5-methoxy-2-[(4-meth-oxy-3,5-dimethyl-2-pyridinyl)methylsulphinyl]-1H-benzimidazole (INN: omeprazole), 5-methoxy-2-[(S)-[(4-methoxy-3,5-dimethyl-2-pyridinyl)methyl]sulphinyl]-1H-benzimidazole (INN: esomeprazole), 2-[3-methyl-4-(2,2,2-trifluoroethoxy)-2-pyridinyl)methylsulphinyl]-1H-benzimidazole (INN: Iansoprazole) and 2-{[4-(3-methoxypropoxy)-3-methylpyridin-2-yl]-methylsulphinyl}-1H-benzimidazole (INN: rabeprazole) and in particular 5-difluoromethoxy-2-[(3,4-dimethoxy-2-pyridinyl)methylsulphinyl]-1H-benzimidazole (INN: pantoprazole) and (−)-5-difluoromethoxy-2-[(3,4-dimethoxy-2-pyridinyl)methylsulphinyl]-1H-benzimidazole [(−)-pantoprazole] may be mentioned by way of example.

The proton pump inhibitors are present as such or in the form of their salts with bases. Examples of salts with bases which may be mentioned are sodium, potassium, magnesium or calcium salts. If the proton pump inhibitors or their salts are isolated in crystalline form, the crystals may contain variable amounts of solvent. Thus, according to the invention, the term “proton pump inhibitor” also includes all solvates, in particular all hydrates, of the proton pump inhibitors and their salts. Pantoprazole-sodium sesquihydrate (=pantoprazole-sodium×1.5 H₂O), (−)-pantoprazole-sodium sesquihydrate, panto-prazole-magnesium dihydrate, omeprazole-magnesium, omeprazole-magnesium tetrahydrate, esomeprazole-magnesium and esomeprazole-magnesium tetrahydrate may be mentioned as particularly preferred salts or hydrates of proton pump inhibitors.

Proton pump inhibitors which may be mentioned for the purposes of the invention in particular are the compounds 5-difluoromethoxy-2-[(3,4-dimethoxy-2-pyridinyl)methylsulphinyl]-1H-benzimidazole (INN: pantoprazole) and (−)-5-difluoromethoxy-2-[(3,4-dimethoxy-2-pyridinyl)methylsulphinyl]-1H-benzimidazole [(-−-pantoprazole]. Particularly preferred salts or hydrates of these proton pump inhibitors that may be mentioned are pantoprazole sodium sesquihydrate (=pantoprazole sodium×1.5 H₂O), pantoprazole magnesium dihydrate and (−)-pantoprazole magnesium dihydrate.

The invention relates in a further aspect to the use of the proton pump inhibitors for the treatment of patents with pathological manifestations induced or influenced by free radicals.

The invention further relates to a method for the treatment of pathological manifestations induced or influenced by free radicals, which consists of administering an effective amount of a proton pump inhibitor to a patient requiring such a treatment.

The invention further relates to the use of the proton pump inhibitors for producing medicinal products for the treatment of pathological manifestations induced or influenced by free radicals.

The invention further relates to a pharmaceutical preparation for the treatment of pathological manifestations induced or influenced by free radicals which contains a proton pump inhibitor as active ingredient.

The invention further relates to a finished medicinal product which comprises a proton pump inhibitor as active ingredient and which contains a reference to the fact that this finished medicinal product can be employed for the treatment of pathological manifestations induced or influenced by free radicals.

### INDUSTRIAL APPLICATION

It is known that a large number of pathological states can be induced or at least influenced by free radicals. Thus, G. Ohlenschläger (Curriculum oncolog. 3/92) for example mentions premature ageing, loss of vitality, a tendency to arteriosclerosis, increase in immunodeficiencies, autoimmune diseases and tendency to tumours. The emphasis in this connection is in particular on those minute alterations and impairments by free radicals which eventually lead to clinical pathological manifestations, such as, for example, impairments of electron transport in mitochondria and in microsomes, molecular oxygen, polyenoic acids in conjunction with transition metals, enzyme reactions and many others.

If free radicals predominate and/or the radical scavenging systems are weak, there is occurrence of mutations, translation errors, crosslinking on macromolecules, membrane damage and lipofuscin formation—and as a result a large number of disorders. Radical reactions proceed like chain reactions (domino effect) over more than a hundred chain members until the reactions are terminated with formation of di- or polymers by formation of oxygen bridges. However, this leads to alterations, dislocations and decompositions of membranes, membrane enzymes and membrane receptors. In addition, the transport mechanisms are destroyed, which may eventually lead to cell death.

Free radicals likewise cause defects in repair systems concerned with repairing the numerous defects in the microstructures of the cell as far as the DNA, and disposing of destroyed structures. If these repair mechanisms are inadequate, it is no longer possible for example for DNA point alterations to be “cut out”. It is thus possible for previously normal gene sections (proto-oncogenes) to become oncogenes—the growth programme of the cell is thus impaired and a possibly no longer controllable proliferation of cells starts.

Lipid peroxidation may be picked out from the large amount of damage to biological structures by free radicals, because precisely this endangers the integrity of the cell as a result of destruction of the cell membrane (double lipid membrane). This event is the basis for most degenerative disorders, starting with rheumatoid disease and extending to Parkinson's disease, multiple sclerosis and other pathological states.

According to the invention, the proton pump inhibitors are employed for treatment of pathological manifestations induced or influenced by free radicals in the form of finished medicinal products. These medicinal products are produced by methods known per se and familiar to the skilled person. The proton pump inhibitors are moreover employed as medicinal products either as such or, preferably, in combination with suitable pharmaceutical excipients or carriers in the form of tablets, coated tablets, capsules, suppositories, patches (e.g. as TTS), emulsions, suspensions or solutions, with the active ingredient content advantageously being between 0.1 and 95%, and it being possible by appropriate choice of the excipients and carriers to obtain a pharmaceutical dosage form which is adapted exactly to the active ingredient and/or to the desired onset of action and/or to the duration of action (e.g. a sustained release form or a gastro-resistant form).

The skilled person is aware, on the basis of his expert knowledge, of which excipients and camers are suitable for the desired pharmaceutical formulations. Besides solvents, gel formers, suppository bases, tablet excipients and other ingredient carriers, it is possible to use for example antioxidants, dispersants, emulsifiers, antifoams, masking flavours, preservatives, solubilizers, colours or, in particular, permeation promoters and complexing agents (e.g. cyclodextrins).

The active ingredients can be administered orally, parenterally or percutaneously.

It has generally proved advantageous in human medicine to administer the proton pump inhibitor in a daily dose of, in particular, 0.1 to 1.5 mg/kg of body weight, where appropriate in the form of a plurality, preferably 1 to 2, of single doses to achieve the desired result. Dosages which can be used for parenteral treatment are similar or (especially on intravenous administration of the active ingredient) usually lower. Every skilled person will easily be able on the basis of his expert knowledge to establish the optimal dosage and mode of administration of the active ingredient which is necessary in each case.

The invention further relates to a pharmaceutical preparation for the treatment of pathological manifestations induced or influenced by free radicals, which comprises in a single dose (tablet, capsule etc.) a proton pump inhibitor as active ingredient in a dose of between 5 and 100, advantageously between 10 and 60, in particular between 20 and 40 mg.

If the proton pump inhibitors are to be employed for the treatment of pathological manifestations induced or influenced by free radicals, the pharmaceutical preparations may also comprise one or more pharmacologically active ingredients of other pharmaceutical groups. Examples which may be mentioned are: tranquilizers (for example from the group of benzodiazepines, e.g. diazepam), spasmolytics (e.g. bietamiverine or camylofin), anticholinergics (e.g. oxyphencyclimine or phencarbamide), local anaesthetics (e.g. tetracaine or procaine), where appropriate also enzymes, vitamins or amino acids.

Combination of the proton pump inhibitors with other drugs which are normally employed for the treatment of pathological manifestations induced or influenced by free radicals should be particularly emphasized in this connection.

Biological Data

1. Introduction

In addition to the established control of acid secretion a second independent anti-inflammatory reactivity of the class of proton pump inhibitors from the pyridyl methyl sulphinyl benzimidazole type was observed in vitro. No detectable effect was seen in the superoxide radical scavenging system. However, an inhibitory reactivity was clearly noticed using three different assays where the highly aggressive hydroxyl radicals were successfully trapped in a concentration dependent manner. There is unequivocal evidence that all benzimidazoles having the sulphoxide group are able to scavenge hydroxyl radicals which were generated during a Fenton reaction. By way of contrast, the corresponding thioethers were substantially less active. In conclusion, pantoprazole has a pronounced inhibitory reactivity towards hydroxyl radicals.

Pantoprazole, a proton pump inhibitor from the benzimidazole type, is successful in the treatment of acid related diseases [1] which is based on the inhibition of the gastric H,K-ATPase [2]. However, acid related disorders are not necessarily associated with increased acid secretion [3].

Acid acts as a noxious agent when physiological conditions have changed e.g during Helicobacter pylori infection, co-treatment using non steroidal antiphlogistic drugs or increased reflux rates of gastric content into the oesophagus. These events are normally accompanied by infiltration of neutrophiles which release oxygen radicals thereby leading to the signs of inflammation [4]. Efforts were undertaken to examine a possible role of proton pump inhibitors to inhibit neutrophile function in releasing superoxide anion O2—, however, they failed to demonstrate clinically significant effects [5,6], because inhibition of PMNs (chemotactic activity and O2—release) function was only in the presence of these inhibitors being 5-50 times higher than transient peak levels achieved in plasma. The sulphoxide moiety and/or the thioether residue in the benzimidazoles were presumed to react with excited oxygen species known to be generated in the course of an inflammatory process. Two prominent excited oxygen species including superoxide and hydroxyl radicals were expected to be scavenged by pyridyl sulphinyl benzimidazoles. In order to monitor this anb-inflammatory reactivity in vitro the inhibition of superoxide radicals was examined using an established superoxide dismutase assay. However, the main focus was directed to the highly reactive hydroxyl radicals. It is well known that these radicals are able to destroy all kinds of biomolecules. They may be generated in vivo and are the cause for a series of diseases including rheumatoid arthritis and other inflammatory processes. Hydroxyl radicals can easily be produced in vitro according to the Fenton reaction with transition metal ions in their low oxidation state, preferentially either with iron(II) compounds or more effectively with copper(I) in the presence of hydrogen peroxide or simply dioxygen [7]. The copper(I)-ions are readily formed upon reacting with ascorbic acid as an electron donor.

Two examples for the Fenton reaction are:
Fe(II)+H2O2→Fe(III)+OH+OH—
Cu(I)+H2O2→Cu(II)+OH+OH—

OH-radicals can also be formed by direct autoxidation of the metal ions in the presence of dioxygen to yield superoxide (O2—) which is spontaneously dismutated to hydrogen peroxide. Thus, the above reactions can proceed.

A suitable assay is the inhibition of the OH-radical-dependent degradation of the heme system of heme proteins [8]. The dedine of the Soret band of the heme group can be conveniently quantified in the 350-450 nm region. An alternative assay used was the depolymerisation of hyaluronic acid by OH-radicals [9] The progressive depolymerisation of this mucopolysaccharide was followed viscosimetrically and should be inhibited accordingly in the presence of the prazoles.

In a third completely different method OH-radicals generated during a Fenton reaction decomposed 2-desoxy-D-ribose [10]. This reaction was monitored by measuring the formation of malondialdehyde-like compounds known to yield coloured thiobarbituric acid adducts which were eventually recorded at 532 nm. In the presence of OH-radical scavengers this dye formabon is progressively inhibited. This was attributed to the extraordinary stability of this compound surviving a hydroxyl radical burst A crucial question was the survival of these benzimidazoles during Fenton-type reactions. These stability measurements were conducted employing HPLC analysis allowing the detection of remaining intact primary compounds.

In order to examine a possible reactivity of the substituted benzimidazoles with superoxide a standard superoxide dismutase assay using nitro tetrazolium chloride was employed [11]. Scavengeing of hydroxyl radicals was measured by three different established methods.

2. Methods

2.1 Degradation of haeme group

Bovine erythrocytes were haemolysed and diluted with water to give an electronic absorption at 408 nm of 1.0. The assay was performed analogous to that of the myeloperoxidase enzymatic activity test where hypochlorite is produced which is known to degrade the heme group of heme proteins [6]. To 574 μl of aqueous haeme solution in a thermostatted cuvette 6 μl of DMF or water and 10 μl of 10 μl aqueous copper sulphate were added for 5 minutes. The reaction was started after the addition of 10 μl 50 μM ascorbic acid and the absorption followed at 37° C. for two minutes in the control experiment. Depending on the solubility the respective inhibitor was added in a volume of 6 μl in DMF or water. The total volume was 600 μl.

2.2.

Depolymerisation of hyaluronic acid

Depolymerisation of hyaluronic acid was measured in terms of viscosity change with time [9]. Potassium hyaluronate was purchased from Sigma-Aldrich Chemie, Steinheim, Germany. All operations were performed at room temperature (22° C.). Relative viscosity was monitored by recording the time (seconds) required for a given volume (0.8 ml) of the reaction mixture to drain by gravity from the barrel of a plastic 1 ml syringe through a needle of appropriate size. The meniscus was timed as it passed between two calibration marks on the syringe barrel. In the case of the benzimidazoles dissolved in DMF the reaction mixture was composed of 1.5 ml hyaluronate (1 mg/ml) and 15 μl 10 mM copper sulphate. The reaction was started with 30 μl of 50 mM ascorbic acid in presence of 10 mM phosphate buffer pH 7.2 and incubated for 5 minutes. The same reaction mixture without the inhibitor compound but in the presence of DMF served as control. When the water-soluble sodium salt of pantoprazole was examined only 2.5 μl 10 mM copper sulphate and 5 μl 50 mM ascorbic acid were needed to yield an appropriate depolymerization effect.

2.3 Thiobarbituric acid assay

The thiobarbituric acid assay was performed according to the method described earlier [10]300 μl 7.5 mM 2-desoxy-D-ribose were added to 1000 μl 10 mM potassium-phosphate buffer, pH 7.4 and incubated for 30 min. at 37° C. in the presence of 100 μl inhibitor compound and 100 μl 10 mM iron(II)ammonium sulphate. The total volume was 1.5 ml. The reaction was stopped by the addition of 1 ml 1% (w/v) thiobarbituric acid dissolved in 50 mM NaOH and 1 ml of 2.8% (w/v) trichloro acetic acid and maintained for 15 min at 95° C. The control reaction mixture contained the same volume of DMFas was present in the inhibitor compound. The dye was measured at 532 nm. A low absorption value indicates that desoxyribose is protected from OH-radical decomposition and vice versa.

2.4 Analysis of benzimidazoles by HPLC

A 200 μl aliquot of the incubation mixture was analysed by HPLC using the analytical column Nucleosil C 8, 5 μm (125×4.6 mm, Fa. Grom, Herrenberg, Germany) and the precolumn was LiCHROPREP RP-2 (12×4 mm, Fa. Merck, Darmstadt, Germany) in a reverse phase. A gradient of acetonitrile (10-45% (v/v)) and 10 mM phosphate buffer, pH 7.4, at a flow rate of 1 ml min-1 was used as effluent on the analytical column.

Results and Discussion

It was of interest to examine pantoprazole as to which degree it is capable to scavenge reactive oxygen species. Pantoprazole did not display detectable reactivity at all in the superoxide radical scavengeing system.

By way of contrast, an inhibitory effect was clearly seen with pantoprazole in three different assays where the highly aggressive hydroxyl radicals were successfully trapped in a concentration dependent manner. The radicals were generated by both the Fe(II)- and the much more potent Cu(II)/ascorbate-mediated Fenton reaction. Biomolecules including the heme system of hemoglobin, hyaluronic acid and desoxyribose they all were effectively destroyed in the presence of this reactive oxygen species. Bleaching of heme was measured by the decrease of the Soret band at 408 nm. Compared to the control 100 μM pantoprazole inhibited the degradation by 75% in this system. In the presence of 50 μM pantoprazole an inhibition of 36% was noticed. Pantoprazole was dissolved in DMF. In the control experiment the same DMF concentration was added to the assay mixture.

To exclude a possible disturbing effect of DMF the water-soluble pantoprazole sodium salt was examined in the same system. Nearly identical results were obtained in this aqueous assay. 100 μM inhibited by 74% and 50 μM by 34%, respectively. Even at a pantoprazole sodium concentration of 25 μM a distinct inhibition was seen.

A similar mode of reaction of pantoprazole was found in the hyaluronic acid depolymerisation assay which was viscosimetrically detected. In the case of 160 μM dissolved in DMF a nearly complete inhibition became apparent, whereas half of this concentration (80 μM) led to 48% of mucopolysaccharide degradation within 20 min.

Surprisingly, in the fully aqueous assay 12 μM of pantoprazole sodium caused a marked inhibition by 46% and 20 μM were needed to protect 90% of the biopolymer. These results encouraged the use of the above aqueous test system as a sensitive tool to monitor the antiinflammatory reactivity at low pantoprazole concentrations.

The stability of pantoprazole under condition of the Fenton reaction was demonstrated in a separate HPLC experiment. The amount of intact compound was analysed after OH-radical exposure. The decrease of pantoprazole was 15% (SD±2.7, n=3 experiments) during 90 min of incubation.

### CONCLUSION

It is intriguing to see that in addition to the established control of acid secretion a second independent reactivity of the class of substituted benzimidazoles is obvious. Acid infiltration into tissue may lead to an attraction of neutrophils and to an undesired inflammatory process. It can be concluded that the proton pump inhibitors from the benzimidazole type may have two issues of therapeutic action; one of inhibition of acid secretion and the other of scavenging hydroxyl radicals and therefore they can be used as anti-inflammatory reagents. The maximal concentration of pantoprazole (Cmax) in plasma after oral administration (5-10 μM) is close to the range of the IC50 values for inhibition of hydroxyl radical generation. The advantage for this approach i.e. inhibition of the product of neutrophils in contrast to inhibition of their function would be not to touch the important immunological and bactericidal function of the neutrophils during infection. These results demonstrate a distinct specificity of pantoprazole as an anti-inflammatory reagent.

### REFERENCES

[1] Fitton A, Wiseman L. Pantoprazole: A review of its Pharmaceutical Properties and Therapeutic Use in Acid-Related Disorders. Drugs 1996; 52: 460-482.
[2] Simon WA, Keeling D J, Laing S M, Fallowfleld C and Taylor A G. BY 1023/SK&F96022: Bio-chemistry of a novel (H+,K+)-ATPase inhibitor. Biochem Pharmacol 1990; 39: 1799-1806.
[3] Moss S F, Arnold R, Tytgat G N J, Spechler S J, Delle fave G, Rosin D, Jensen R T, Modlin I M, Consensus Statement for Management of Gastroesophageal Reflux Disease. J Clin Gastroenterol 1998; 27:6-12.
[4] Naya M J, Pereboom, Ortego J, Alda J O, Lanas A. Superoxide anions produced by inflammatory cells play an important part in the pathogenesisi of acid and pepsin induced oesophagitis in rabbits. GUT; 40: 175-181
[5] Wandall J H. Effects of omeprazole on neutrophile chemotaxis, supeoxide production, degranulation, and translocation of cytochrome b-245 1992; 33:617-621
[6] Capodicasa E, De bellis F, Pelli M A. Effect of Lansoprazole on Human Leukozyte Function. Immunopharmacology and Immunotoxicology 1999; 21:357-377.
[7] Halliwell, B. and Gutteridge, J. M. C. Oxygen toxicity, oxygen radicals, transition metals and disease. Biochem. J. 1984; 219: 1-14.
[8] Albrich, J. M., McCarthy, C. A. and Hurst, J. K. Biological reactivity of hypochlorous acid: Implication for microbial mechanisms of leukocyte myeloperoxidase. Proc. Natl. Acad. Sci. USA 1981; 78: 210-214.
[9] Mc Cord J M,. Free radicals and inflammation; Protection of Synovial Fluid by Superoxide Dismutase. Science 1974; 185:529-531
[10 Gutteridge, J. M. C. Thiobarbituric acid-reactivity following iron-dependent free-radical damage to amino acids and carbohydrates. FEBS Lett. 1981; 128: 343-346.
[11] Gartner A, Hartmann H J, Weser U. A simple, rapid and efficient isolation of erythrocyte Cu2Zn2-superoxide dismutase. Biochem J, 1984: 221: 549-551.

1. (canceled)

2. (canceled)

3. A method for the treatment of pathological manifestations induced or influenced by free radicals, consisting of administering an effective amount of a proton pump inhibitor or its pharmacologically acceptable salt to a patient requiring such a treatment.

4. (canceled)

5. (canceled)

6. The method according to claim 3, wherein the active ingredient is selected from the group consisting of leminoprazole, nepaprazole, tenatoprazole, omeprazole, esomeprazole, lansoprazole, rabeprazole and pharmacologically acceptable salts thereof.

7. The method according to claim 3, wherein the active ingredient is pantoprazole or a pharmacologically acceptable salt thereof.

8. The method according to claim 3, wherein the active ingredient is (S)-pantoprazole [(−)-pantoprazole] or a pharmacologically acceptable salt thereof.

9. The method according to claim 3, wherein the pathological manifestation induced or influenced by free radicals an inflammatory disease.

10. The method according to claim 3, wherein the pathological manifestation induced or influenced by free radicals a rheumatic disorder.

11. The method according to claim 3, wherein the pathological manifestation induced or influenced by free radicals an uncontrolled proliferation of cells.

12. The method according to claim 3, wherein the pathological manifestation induced or influenced by free radicals, Parkinson's disease.

13. The method according to claim 3, wherein the pathological manifestation induced or influenced by free radicals multiple sclerosis.
